(19) United States
(12) Patent Application Publication
(10) Pub. No.: US 2023/0012468 A9
(43) Pub. Date: Jan. 12, 2023

(54) Treatment Application Garment

(71) Applicant: Snails, LLC, Bedford, IN (US)

(72) Inventor: Tammie S. Biddle, Bedford, IN (US)

(21) Appl. No.: 16/585,673

(22) Filed: Sep. 27, 2019

Related U.S. Application Data
(63) Continuation-in-part of application No. 15/899,164, filed on Feb. 19, 2018, now abandoned.

(51) Int. Cl. A61M 35/00; A61K 8/35; A61Q 3/02; A61K 8/365; A61Q 3/04; A45D 29/00

(52) U.S. Cl. CPC A61M 35/10; A61K 8/35; A61Q 3/02; A61K 8/365; A61Q 3/04; A45D 29/00; A61M 2205/0216; A61K 2800/87; A61M 2210/083; A61M 2210/086; A61M 2209/06; A45D 2200/1018; A45D 2200/1036

(57) ABSTRACT

A treatment application garment, such as a glove or a sock, is disclosed. The garment allows for various treatments directed toward the fingers and toes, such as the fingernails and toenails. For example, each digit of the garment may contain an absorbent pad containing acetone. The hand or foot is inserted into the garment such that the nail is positioned below the absorbent pad containing the acetone, allowing the acetone to be constantly applied to the nail to remove polish therefrom. After the treatment is complete, the glove may be removed and the open end thereof tied off, thereby containing the acetone and the smell thereof within the glove. Other features and treatments are also disclosed.

### TECHNICAL FIELD

This patent application relates to garments and, more particularly, to a treatment application garment.

### BACKGROUND OF THE DISCLOSED EMBODIMENTS

Manicures using gel polish, which gel polish is normally a water solution of acrylic polymer, have become very popular because of the high durability of the gel polish. Another benefit of a gel manicure is the speed and convenience of application of the polish to the nails. Use of a gel polish gives a brilliant manicure in a fraction of the time that it takes for a traditional manicure; it lasts considerably longer; and it is almost indestructible.

Removing the gel polish is complicated and time consuming, requiring a long, messy, four-step process. First, a cotton ball is soaked in pure acetone. Then, the acetone-soaked cotton ball is placed in the center of a piece of cut aluminum foil and placed on top of the gel polish to be removed. Next, the aluminum foil is wrapped around the fingertip to hold the cotton ball in position on the nail. This procedure is repeated for each of the other fingernails. The cotton balls are left in place for 15 minutes, after which each foil is grasped and twisted around the fingernail to rub off as much of the chemically compromised polish as possible. The foil tips are then removed from the fingers, which foil tips can scratch and cut into the fingers during removal. Any remaining polish on the fingernail is then removed by rubbing, such as with a rubber-ended cuticle pusher.

This procedure has the following disadvantages: the difficulty and discomfort caused by working with the aluminum foil; it is nearly impossible to fit the aluminum foil securely around the finger tips; acetone easily runs out of the aluminum foil, down the user's hand and onto adjacent surfaces; the user cannot use her hand during the removal process; and, a user without a helper can only remove polish from one hand at a time (using a minimum of 15 minutes per hand).

Therefore, there exists a need in the art for a more convenient method for removing gel polish. Further, there exists a need in the art for a method for removing gel polish that allows the user to use her hands during the procedure.

### SUMMARY OF THE DISCLOSED EMBODIMENTS

In one embodiment of the disclosure, a treatment application garment is disclosed, comprising: a body with a plurality of integrally extending digits; at least one sealed pocket, wherein each of the at least one sealed pockets is disposed in a respective one of the plurality of integrally extending digits; a liquid disposed in each of the at least one sealed pockets; and at least one absorbent pad, wherein each of the at least one absorbent pads is disposed adjacent to a respective one of the at least one sealed pockets.

In some embodiments of the disclosure, the body with a plurality of extending digits comprises a glove.

In some embodiments of the disclosure, the body with a plurality of extending digits comprises a sock.

In some embodiments of the disclosure, the body with a plurality of extending digits is formed from a liquid impermeable material.

In some embodiments of the disclosure, the body with a plurality of extending digits is formed from a material selected from the group consisting of: polyethylene, polypropylene, polyvinyl chloride (PVC), synthetic rubber, natural rubber, neoprene, polyacrylonitrile, or silicone (polysiloxane).

In some embodiments of the disclosure, at least a portion of each of the plurality of extending digits is formed from a liquid impermeable material.

In some embodiments of the disclosure, the liquid comprises acetone.

In some embodiments of the disclosure, each of the at least one sealed pockets is attached to a respective one of the plurality of integrally extending digits.

In some embodiments of the disclosure, each of the at least one sealed pockets comprises an area of weakened material.

In some embodiments of the disclosure, the area of weakened material comprises a line of weakened material.

In some embodiments of the disclosure, each of the at least one sealed pockets is formed from a first material and each of the plurality of digits is formed from a second material, wherein the first material is weaker than the second material.

In some embodiments of the disclosure, the treatment application garment further comprises: a first tab extending from the body; and a second tab extending from the body.

In some embodiments of the disclosure, the treatment application garment further comprises: a first pocket formed in the body, the first pocket having a first end and a second end; a first opening formed through the body at the first end; a second opening formed through the body at the second end; a second pocket formed in the body, the second pocket having a third end and a fourth end; a third opening formed through the body at the third end; a fourth opening formed through the body at the fourth end; and a ribbon of material extending from a position external to the body, through the first opening, through the first pocket, through the second opening, through the third opening, through the second pocket, and through the fourth opening.

In some embodiments of the disclosure, the treatment application garment further comprises an elastic material coupled to the body and substantially encircling the body.

In some embodiments of the disclosure, the liquid comprises a nail conditioning treatment.

In some embodiments of the disclosure, the nail conditioning treatment comprises glycolic acid.

In some embodiments of the disclosure, the liquid comprises a nail fungus treatment.

In some embodiments of the disclosure, the nail fungus treatment comprises a material selected from the group consisting of: bleach, snakeroot extract, tea tree oil, oregano oil, olive leaf extract, ozonized oil, ozonized olive oil, ozonized sunflower oil, and vinegar.

In some embodiments of the disclosure, at least one of the plurality of extending digits is formed from a touchscreen compatible material.

### BRIEF DESCRIPTION OF THE DRAWINGS

The embodiments and other features, advantages and disclosures contained herein, and the manner of attaining them, will become apparent and the present disclosure will be better understood by reference to the following description of various exemplary embodiments of the present disclosure taken in conjunction with the accompanying drawings, wherein:

FIG. 1 is a plan view of a treatment application garment according to one embodiment of the present disclosure;

FIG. 2 is a partial cross-sectional view of the treatment application garment of FIG. 1;

FIG. 3 is a plan view of a treatment application garment according to one embodiment of the present disclosure mounted upon a user's hand;

FIG. 4 is a partial cross-sectional view of the treatment application garment of FIG. 3;

FIG. 5 is a plan view of a treatment application garment according to one embodiment of the present disclosure;

FIG. 6 is a partial plan view of a treatment application garment according to one embodiment of the present disclosure;

FIG. 7 is a partial plan view of a treatment application garment according to one embodiment of the present disclosure;

FIG. 8 is a cross-sectional view of the treatment application garment of FIG. 7; and

FIG. 9 is a partial plan view of a treatment application garment according to one embodiment of the present disclosure.

### DETAILED DESCRIPTION OF THE DISCLOSED EMBODIMENTS

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

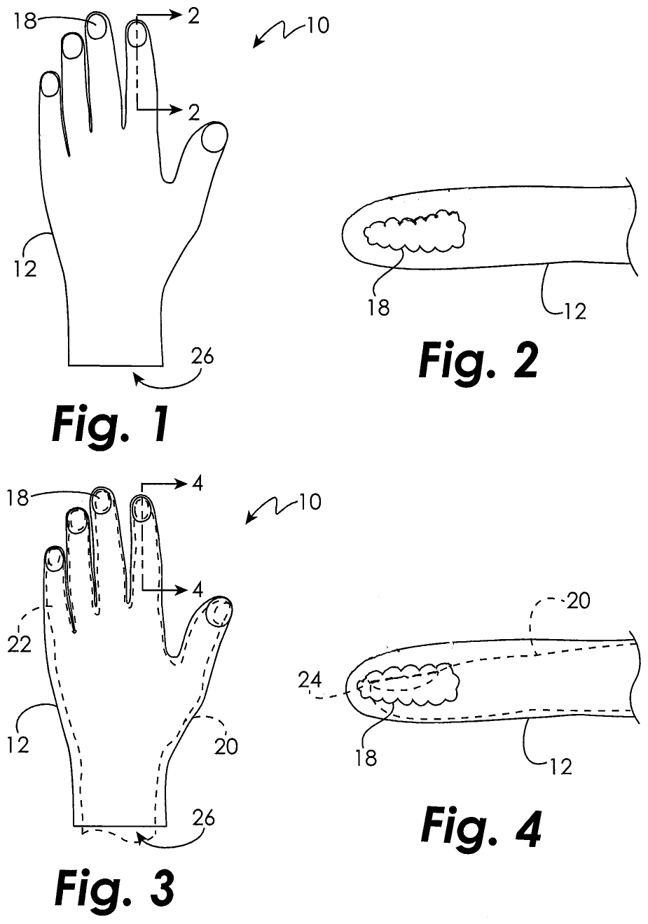

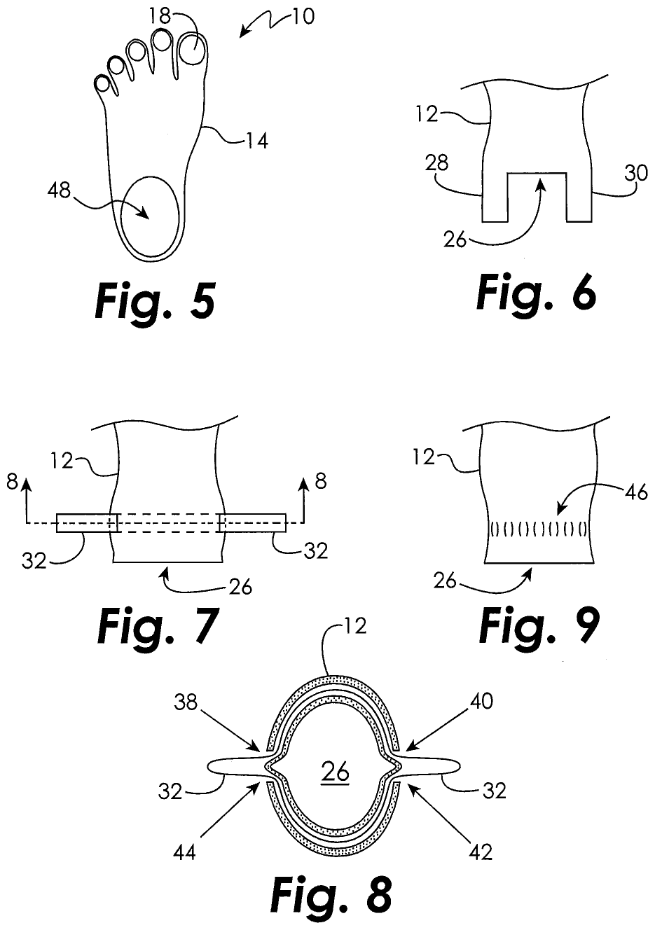

Referring now to FIGS. 1-4, a treatment application garment useful for removing nail polish and for other cosmetic, therapeutic, medical, or other classes of treatment is illustrated in accordance with an embodiment of the present disclosure and indicated generally at 10. In the embodiment of FIGS. 1-4, the treatment application garment 10 is illustrated as a glove 12, formed as a body with a plurality of integrally extending digits, to be fitted over a user's hand. In other embodiments, the treatment application garment may be in the form of a sock 14, formed as a body with a plurality of integrally extending digits, to be fitted over a user's toes and foot as illustrated in FIG. 5, to name just one non-limiting example. It will therefore be appreciated that, while the treatment application garment 10 is described hereinbelow as a glove for illustrative purposes, references to fingers, fingernails, fingertips and the like should be interpreted to encompass any digit, such as toes, toenails, toe tips, and the like.

With continuing reference to FIGS. 1-4, although treatment application garment 10 is illustrated in the form of a glove 12, it will be appreciated from the present disclosure that the treatment application garment 10 may also comprise a pair of gloves 12 in some embodiments, with one glove in the pair being configured for the user's left hand and the other glove in the pair being configured for the user's right hand. In some embodiments, the treatment application garment 10 comprises a glove 12 formed from a material that is substantially impermeable to liquids. For example, the glove 12 may be formed from a polymer, either natural or synthetic in some embodiments, such as polyethylene, polypropylene, polyvinyl chloride (PVC), synthetic rubber, natural rubber, neoprene, polyacrylonitrile, or silicone (polysiloxane), to name just a few non-limiting examples. Other non-liquid permeable materials may also be used. In some embodiments, the glove 12 comprises a non-liquid permeable material in the region of the user's fingertips, while the remainder of the glove may comprise another material.

The glove 12 is formed with an absorbent pad 18, such as a cotton ball (natural or synthetic), paper, sponge (natural or synthetic) or other absorbent material, disposed in each fingertip, which absorbent pad 18 contains (e.g., has been soaked in) acetone in one embodiment. Acetone is effective in removing nail polish from the fingernail, but other non-acetone nail polish removers may also be used. The material from which the glove 12 is made must be compatible with the contents placed in each fingertip. Therefore, for embodiments in which the absorbent pad 18 contains acetone, the glove 12 may be formed from polyethylene, which is impervious to acetone. In other embodiments discussed hereinbelow, the absorbent pad 18 may contain other materials. With particular reference to FIGS. 3-4, when a user's hand 20 is positioned inside the glove 12, each finger 22 is positioned under the absorbent pad 18, such that the absorbent pad 18, and any contents of the absorbent pad 18, contacts and substantially surrounds the user's fingernail 24.

The absorbent pad 18 will direct the acetone (or other treatment material) to the user's fingernail 24 and keep the acetone in contact therewith. After 15 minutes, or other desired waiting time, the glove 12 may be removed and any remaining polish may be removed by conventional means.

After removal of the glove 12, the open proximal end 26 of the glove 12 may be tied in a knot, thereby containing the acetone liquid and the odor thereof inside the glove 12 during and after disposal of the glove 12. In order to facilitate tying of the open proximal end 26 of the glove 12, extending tabs 28 and 30 may be formed to extend from the open proximal end 26 in an embodiment, as shown in FIG. 6. The tabs 28 and 30 may be tied in a knot, thereby closing the open proximal end 26. In another embodiment, the glove 12 may be provided with a continuous ribbon of material 32 that is partially disposed within pockets 34 and 36 formed within the glove 12 distal to the proximal open end 26, and partially disposed outside of the glove 12 by means of exiting the glove 12 at openings 38, 40, 42 and 44 formed therein, as illustrated in FIGS. 7-8. In another embodiment, the glove 12 may be formed with an elastic material 46 disposed at the proximal open end 26, or distal thereof, as shown in FIG. 9, in order to provide a snug fit to the user's wrist. It will be appreciated from the present description that the closures of FIGS. 6-8 may be provided in addition to the elastic material 46 in the same glove 12 in some embodiments.

With reference to FIG. 5, it will be appreciated from the present disclosure that the features of the sock 14 are analogous to those of the glove 12. That is, the toes for holding absorbent pads 18 containing acetone (or other liquids), and optional tabs 28, 30, ribbon material 32, pockets 34 and 36, openings 38, 40, 42 and 44, and/or elastic 46 provided at or near the proximal open end 48 thereof.

It will be appreciated from the present description that the absorbent pads 18 may contain liquids or gels other than acetone, such as non-acetone nail polish removers, to name just one non-limiting example. In some embodiments, the absorbent pads 18 may contain a nail conditioning treatment, such as glycolic acid to help slough away any separated nail cells that can lead to flaking and breakage and to rehydrate the nails, to name just one non-limiting example. In other embodiments, the absorbent pads 18 may contain a nail fungus treatment, such as bleach, snakeroot extract, tea tree oil, oregano oil, olive leaf extract, ozonized oils such as olive oil and sunflower oil, and/or vinegar, to name just a few non-limiting examples. It will be appreciated from the present disclosure that the treatment application garment disclosed herein may be used for other cosmetic, therapeutic, medical, or other classes of treatments.

In some embodiments, after the absorbent pads 18 and acetone (or other treatment material) is added to each fingertip, the glove 12 may be placed in a package (not shown) and the package may be sealed with an airtight seal to prevent evaporation of the acetone (or other treatment material) during transport and/or storage of the glove 12 prior to use. In other embodiments, the open proximal end 26 of the glove may be sealed, such as by heat sealing, to name just one non-limiting example, to prevent evaporation of the acetone (or other treatment material) therein. Such seal may then be removed by the user prior to use.

Use of the treatment application garment 10, whether in the form of a glove 12 or a sock 14, offers several advantages over the prior art aluminum foil procedure. Use of the treatment application garment 10 is simple and easy; the garment 10 stays on securely and prevents dripping of the acetone onto adjacent surfaces; the garment 10 allows removal of polish from both hands at the same time; the garment 10 allows use of the user's hands during the removal process (even use of a device with a touchscreen, so long as the material from which the garment 10 is constructed is touchscreen compatible); the garment 10 uses a controlled and pre-measured amount of acetone; the garment 10 contains the mess and smell of the acetone, tying up the glove after use contains the smell in the trash; and use of the garment 10 permits a nail care professional to treat multiple clients' digits simultaneously.

While the disclosure has been illustrated and described in detail in the drawings and foregoing description, the same is to be considered as illustrative and not restrictive in character, it being understood that only certain embodiments have been shown and described and that all changes and modifications that come within the spirit of the disclosure are desired to be protected.

What is claimed is:

1. A treatment application garment, comprising:
a body with a plurality of integrally extending digits;
at least one absorbent pad, wherein each of the at least one absorbent pads is disposed in a respective one of the plurality of integrally extending digits; and
a liquid disposed in each of the at least one absorbent pads.

2. The treatment application garment of claim 1, wherein the body with a plurality of extending digits comprises a glove.

3. The treatment application garment of claim 1, wherein the body with a plurality of extending digits comprises a sock.

4. The treatment application garment of claim 1, wherein the body with a plurality of extending digits is formed from a liquid impermeable material.

5. The treatment application garment of claim 1, wherein the body with a plurality of extending digits is formed from a material selected from the group consisting of: polyethylene, polypropylene, polyvinyl chloride (PVC), synthetic rubber, natural rubber, neoprene, polyacrylonitrile, or silicone (polysiloxane).

6. The treatment application garment of claim 1, wherein at least a portion of each of the plurality of extending digits is formed from a liquid impermeable material.

7. The treatment application garment of claim 1, wherein the liquid comprises acetone.

8. The treatment application garment of claim 1, further comprising:
a first tab extending from the body; and
a second tab extending from the body.

9. The treatment application garment of claim 1, further comprising:
a first pocket formed in the body, the first pocket having a first end and a second end;
a first opening formed through the body at the first end;
a second opening formed through the body at the second end;
a second pocket formed in the body, the second pocket having a third end and a fourth end;
a third opening formed through the body at the third end;
a fourth opening formed through the body at the fourth end; and
a ribbon of material extending from a position external to the body, through the first opening, through the first pocket, through the second opening, through the third opening, through the second pocket, and through the fourth opening.

10. The treatment application garment of claim 1, further comprising an elastic material coupled to the body and substantially encircling the body.

11. The treatment application garment of claim 1, wherein the liquid comprises a nail conditioning treatment.

12. The treatment application garment of claim 1, wherein the nail conditioning treatment comprises glycolic acid.

13. The treatment application garment of claim 1, wherein the liquid comprises a nail fungus treatment.

14. The treatment application garment of claim 1, wherein the nail fungus treatment comprises a material selected from the group consisting of: bleach, snakeroot extract, tea tree oil, oregano oil, olive leaf extract, ozonized oil, ozonized olive oil, ozonized sunflower oil, and vinegar.

15. The treatment application garment of claim 1, wherein at least one of the plurality of extending digits is formed from a touchscreen compatible material.

16. The treatment application garment of claim 1, further comprising a package into which the body with a plurality of integrally extending digits is placed, and an airtight seal formed on the package after the body with a plurality of integrally extending digits is placed into the package.
